(19) United States
(12) Patent Application Publication
(10) Pub. No.: US 2005/0234498 A1
(43) Pub. Date: Oct. 20, 2005

(54) Dilatable balloon implant

(76) Inventors: Dietrich Gronemeyer, Sprockhovel (DE); Matin Deli, Mulheim (DE); Jurgen Speder, Bochum (DE); Jorn Richter, Munster (DE)

Correspondence Address: REED SMITH LLP, 3110 FAIRVIEW PARK DRIVE, SUITE 1400, FALLS CHURCH, VA, 22042, US

(21) Appl. No.: 10/515,479

(22) PCT Filed: May 23, 2003
(86) PCT No.: PCT/EP03/05407

§ 371 Date: May 10, 2005

(30) Foreign Application Priority Data
May 25, 2002 (DE) 10223332,2

(51) Int. Cl. A61F 2/44; A61F 2/04

(52) U.S. Cl. 606/192; 623/23.67; 623/17.12

(57) ABSTRACT

The invention relates to a dilatable balloon implant, which is configured with a limited permeability to liquid. The invention also relates to a vertebroplasty device comprising a balloon implant with a limited permeability to liquid, which is connected to the distal end of an introduction sleeve in such a way that the interior of said implant communicates with the introduction sleeve lumen.

The invention relates to a dilatable balloon implant. The invention also relates to a device for placing such an implant in bone cavities.

A known method for treating vertebra factures (caused in particular as a result of osteoporotic changes in bone tissue) consists in directly filling bone cement or another filler material via a percutaneous access tract into the vertebra and stabilizing it in this manner (vertebroplasty). This method has a disadvantage in that the vertebra cannot be erected prior to stabilization. Moreover, this method involves the risk of filler material exiting from the inner vertebra cavity and entering the body.

Moreover, filler material may also enter the spinal channel, the neuroforamen or the venous plexus of the vertebra and obstruct the latter or get carried away with the bloodstream, thus causing embolies or infarctuses. This method involves the particular problem that the risk of material exiting the vertebra cavity increases if the viscosity of the filler material is too low, while—in the case of the viscosity being too high—the material hardens too quickly, which in turn leads to an inadequate filling.

According to a further state-of-the-art treatment method, the spongiosa of the vertebra is compressed and thus expanded, using one or several balloon catheters (cyphoplasty), before the cavity is filled with filler material. This method serves, on the one hand, to seal cracks in order to prevent the potential escape of filler material and, on the other hand, to potentially erect the vertebra before the filler material is introduced. The need, associated with this method, to remove the balloon catheter extends the length of the surgical intervention and holds the risk of the tonus of the back muscles undoing a previously performed erection. Moreover, cyphoplasty cannot completely prevent filler material entering the spinal channel, a neuroforamen or the venous plexus.

In view of the problems connected with the state-of-the-art treatment of vertebra fractures, the objective of the invention is to provide an implant that minimizes the risk of filler material exiting during vertebroplasty while keeping the intervention time as short as possible and which enables the erection of the vertebra.

According to the invention, this objective is achieved by the use of a balloon implant of the type described above, which provides limited liquid permeability.

The balloon implant according to the invention is introduced, in deflated condition, into the prepared (i.e. opened) vertebra through a percutaneous access tract using a tube-shaped introduction sleeve. Once placed inside the vertebra, the balloon implant is dilated, preferably by introducing a filler material through the introduction sleeve. The limited liquid permeability ensures, on the one hand, that the risk of filler material entering the spinal channel is minimized and enables, on the other hand, a safe connection to be achieved between the implant and the bone. This permits the use of filler material with a lower viscosity than is used in state-of-the-art treatment methods, thus eliminating also the risk of the filler material hardening before the vertebra cavity is completely filled. Moreover, the pressure exerted as the balloon is dilated permits the vertebra to be erected, if so desired.

After the cavity has been filled, the proximal inflow opening of the implant is closed, and the implant is separated from the introduction sleeve.

The use of the implant as described by this invention leads to a better therapy result than that obtained with state-of-the-art operation methods in that it minimizes the complications related to excessive leakage of filler material and overly long intervention times.

In its simplest embodiment, the implant consists only of the balloon proper, though it may also be provided with further elements (e.g. joining elements). In this case, only the balloon proper needs to have limited liquid permeability.

The balloon may consist of a material that has limited liquid permeability or of a liquid impermeable material that is provided with pores that impart limited liquid permeability to the balloon.

The materials may be elastic or non-elastic, provided they meet the requirements for medical materials in terms of physiological compatibility and tear resistance. In a preferred embodiment, plastic materials are used, which can be kept particularly small for introduction into the cavity, so that they can be used for instance with catheters that have a particularly small cross section.

In a preferred embodiment, the liquid perrneable material is a textile tissue or fabric, notably a nylon tissue. Materials that meet the requirements of DIN Standard 53861-3 are especially suitable. The density of the balloon material used must allow a limited amount of liquid to leak out before the filler material hardens. A competent expert can easily select a suitable balloon material for the specific filler material employed.

A material that is particularly suitable as liquid impermeable material with pores is perforated or stippled latex, as that material is, in general, physiologically tolerable and elastic.

The size and number of pores (i.e. the pore density) of the inherently liquid impermeable material are dependent on the filler material used (e.g. conventional bone cement or polymerizing filler materials) and can be easily determined for a given filler material by a competent expert. For customary materials, a pore density of up to 5%, preferably 1 to 3%, related to the material surface, is especially suitable.

The pores are to be so dimensioned that, during therapy, bone filler material exits an amount of 0.05 to 6, preferably 1 to 4, and especially preferably 2 to 3%, thus ensuring proper anchorage of the implant due to a high number of pores.

Materials (that are inherently water permeable or inherently water impermeable, but provided with pores) are especially suitable as balloon materials, if they guarantee permeability of the balloon to medical bone filler material in the range of 0.05 to 6, preferably 1 to 4 and especially preferably 2 to 3%.

These permeability levels denote the amount of filler material introduced in liquid condition that exits during the time until the filler material has hardened inside the cavity, the total amount of filler material introduced into the balloon being 100%. Thus the material to be selected by the expert depends on the filler material to be used and its viscosity. It is no problem for an expert to determine the best suitable balloon material for the filler material used (lower viscosity or slower hardening rate—higher material density).

The implant according to the invention permits the use of filler material of particularly low viscosity (In which case a denser material must be used, so that the appropriate permeability levels as mentioned above can be achieved), which significantly reduces the risk of filler material hardening before the intervention is complete and also permits the use of small-bore introduction sleeves and other small-bore devices. Such low permeability levels minimize the operation risk related to excessive leakage of filler material, while permitting the implant to be securely fixed inside the vertebra cavity.

In non-dilated condition, the balloon assumes a bag-like shape. In an expedient embodiment, it assumes, in dilated condition and without the action of external constraints (e.g. the shape of the vertebra cavity), a predetermined form, which may be essentially ball-shaped or cuboid-shaped or even of more complex shape. Thus, for any vertebra configuration, a specifically adapted implant can be chosen, which safely ensures that the cavity is completely filled. This safety is enhanced by the use of elastic balloon materials, which guarantee a particularly good adaptation of the dilated balloon shape to the shape of the cavity.

In another expedient embodiment, the implant—for the purpose of being separated from the introduction means—is provided with a thinner wall area at its proximal, open end, which functions as a predetermined breaking point. This especially low-cost variant enables the balloon to be closed and the implant to be separated from the introduction device by means of a combined turning/slight pulling motion after the cavity has been filled, so that the implant can be easily separated due to its pull reaction. (In this case, the access tract to the vertebra cavity should have a smaller cross section than the cavity itself, so that the implant, after being filled in, is kept safely in place by a mechanical clamping action even before the material has hardened.)

In a further expedient embodiment, the implant is provided, in its proximal area, with a joining element, which permits a separable and conductive connection to be formed between the implant and its introduction sleeve. Depending on what type of joining element is used, the implant is separated from the introduction sleeve from outside, e.g. by mechanical action, electrolytic detachment or by means of laser flashes. Such joining elements and the materials required to make them are known to competent experts.

Yet a further expedient embodiment is a design, in which the implant is provided with a closing device for the proximal opening, which the surgeon can operate from outside (e.g. a plug or a loop that can be closed by exerting a pulling action).

The invention relates also to a vertebroplasty device with a balloon implant according to any of the above claims, which is connected to the distal end of an introduction sleeve, so that its interior communicates with the introduction sleeve lumen. (Here the term “distal end” does not necessarily mean the distal tip of the introduction sleeve, but the distal end area.)

The implant can basically be joined to the introduction sleeve using any of the known expedient techniques—thus the implant may be joined to the introduction sleeve at the latter's external or internal circumference or the connection may take the form of a continuous transition from the implant to the introduction sleeve.

An expedient solution is a device, in which the balloon is joined to the introduction sleeve by a form-fit joining method, such as gluing, clamping expanding or bolting if necessary, the balloon and/or the introduction sleeve is/are to be provided with suitable retaining elements.

Separability between the implant and the introduction sleeve is ensured either by a connection between the introduction sleeve and the balloon that is itself separable (e.g. the above-mentioned bolting or clamping methods or electrolytic separability of one or several joining elements) or by an inseparable connection between the introduction sleeve and the balloon, with the balloon being provided with a predetermined breaking point (see above).

An expedient device is a type of device, in which the open end of the balloon is secured to the inner circumference of the introduction sleeve.

According to a further, especially expedient embodiment, the open end of the balloon is pulled over the distal end of the introduction sleeve and secured to the outer circumference of the introduction sleeve.

In an especially advantageous embodiment of the device, the introduction sleeve is provided, at its distal end, with a reinforcing element that serves to strengthen the connection between the distal end of the introduction sleeve end and the proximal end of the implant. It may be part of the introduction sleeve wall (turned down distal end) or take the form of an additional element (e.g. a ring), which is firmly connected to the introduction sleeve in a conventional manner.

The element is preferably designed as a clamp or press ring, which, acting as a stopper bead, firmly clamps the proximal end of the balloon to the introduction sleeve (thus providing an additional connection to the outer wall) or locks it firmly in place (by pressing it against the inner wall).

The introduction sleeve is preferably made of medical grade steel. All physiologically tolerable materials of high strength and resistance are, in general, suitable for this purpose.

In an especially preferred embodiment, the introduction sleeve is guided through the hollow needle of a medical trocar to facilitate the introduction.

The invention is hereinafter explained in more detail based on the exemplary embodiments shown in the figures.

Reference is made to the following figures:

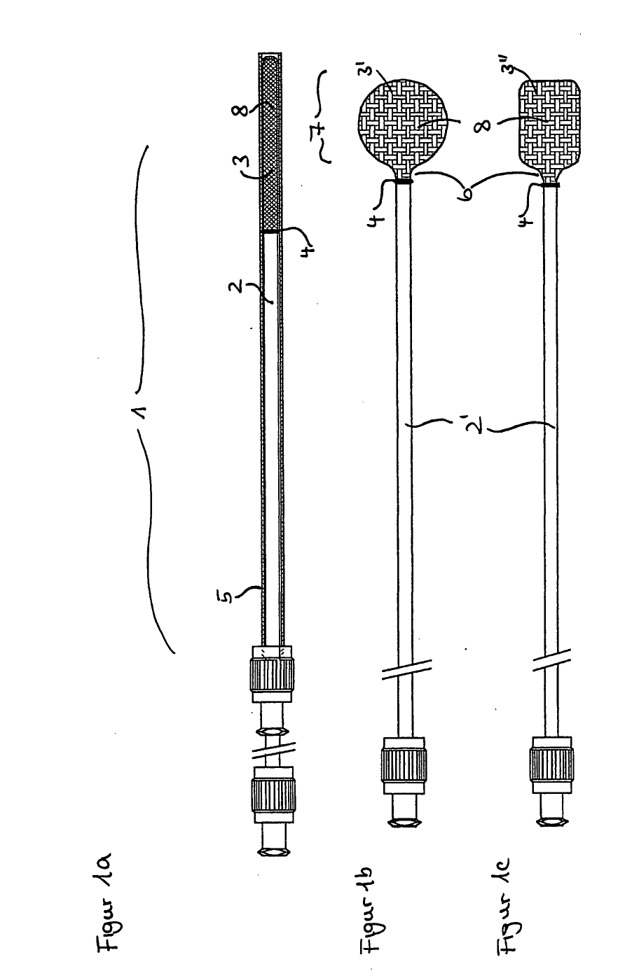

FIG. 1a is a not-true-to-scale representation of a vertebroplasty device 1 with a catheter 2 prior to the introduction into the cavity

FIG. 1b is a not-true-to scale representation of a vertebroplasty device 1 with a ball-shaped dilated balloon implant 3

FIG. 1c is a not-true-to-scale representation of a vertebroplasty device 1 with a cuboid-shaped dilated balloon implant 3

The vertebroplasty device 1 shown in FIG. 1a comprises an introduction sleeve 2 and a dilatable balloon implant 3. At the distal end of the introduction sleeve 2, the balloon implant 3 is attached to the outer wall of the introduction sleeve 2 in a form-fit manner by hot-forming. To provide additional safety, the connection is reinforced by a clamping ring designed as a stopper bead 4 that acts on the outer circumference of the introduction sleeve 2 to prevent any movement towards the distal end.

After establishing a percutaneous access tract and opening the vertebra using known techniques, e.g. a trocar, the introduction aid 5, e.g. the hollow needle of the trocar, is moved up to the vertebra opening, and the balloon implant 3 is introduced into the vertebra cavity in deflated condition.

In this x-ray-controlled intervention, the introduction sleeve 2 is first moved up to the vertebra opening. Then the balloon is introduced into the cavity by moving the introduction sleeve 2 forward, making sure that the distal end of the introduction sleeve 2 is introduced as well to ensure complete introduction. The intervention is x-ray-controlled, the positions of the introduction aid or catheter 5 and the introduction sleeve 2 being controllable due to the use of markers.

Subsequently a suitable filler material (polymethyl methacrylate, bone cement or another suitable, preferably x-ray-resistant material) is introduced from outside through the introduction sleeve 2 into the balloon implant 3, placed inside the cavity, until the implant fills the cavity.

FIGS. 1b and 1c show two different embodiments of the implant 3′/3″, which may assume different shapes in dilated condition (ball 3′ or cuboid 3′″). Thus, depending on the shape of the cavity, an implant 3 can be chosen that is designed to assume a shape that is particularly well adapted to the form of the cavity. Moreover, due to the elasticity of the nylon material, the implant 3 when dilated will expand beyond its predetermined shape, advancing further into the cavity, thus filling it particularly well.

In this example, the implant 3 consists of the balloon proper. The balloon is so preshaped that, in dilated condition, it has a neck 6 (smaller outer circumference compared to the body 7) and a body 7. In the area of the neck 7, the implant 3 is provided with a thinner wall section, owing to which the implant 3, after being filled with the filler material, can be separated from outside from the introduction sleeve 2 by turning the introduction sleeve 2 to some degree while exerting a slight pull.

The turning movement, in combination with the elasticity of the nylon material 8 used for the implant 3, causes the neck 6 of the implant 3 to close, so that no major quantities of filler material can exit towards the proximal end.

Moreover, suitable closing means for the cavity (e.g. plugs or bolts) may be used to close the cavity after it has been filled. Alternatively, implants may be used that are themselves equipped with closing elements (not shown).

In addition, after having performed the turning movement that brings about the closure of the neck 6 and separates the implant 3 (by the pull action and a further turning movement), the surgeon may for instance wait until the filler material has hardened. Here it must be made sure that the distal end of the introduction sleeve 2 is removed from the vertebra and placed at the opening of the cavity before the hardening process is complete. In this manner, it is ensured that no accidental connection can form between the introduction sleeve 2 and the vertebra.

In these embodiments, the introduction aid 5 is designed as a hollow needle of a medical trocar the introduction sleeve 2, together with the implant 3, is moved through the introduction aid 5 into the therapy position.

First, the ensemble consisting of the introduction aid and obturator (trocar) is introduced through the skin into the bone in an x-ray-controlled process. Once the target position has been reached, the obturator is pulled out. The hollow needle now constitutes the working tract leading to the target location. Through the hollow needle, the soft balloon can be pushed forward into the therapy position without the risk of sticking to the bone skin while being pushed forward. (FIG. 1a above shows the introduction aid pushed over the hollow needle and balloon.) Once the balloon is in its target position, the introduction aid is pulled back to such an extent that the balloon can be dilated by injecting bone cement.

The filler material and the balloon material are to be so selected to ensure that 2-3% of the material exits into the cavity before the filler material is hardened. This makes sure that the implant is safely placed inside the cavity without the risk of filler material getting into the posterior quarter of the vertebra and filling venous vessels in that area (or the risk of embolies and infarctuses being caused by freely floating, hardened filler material).

Depending on the configuration of the cavity, it may be expedient to introduce several implants, using different access tracts, where appropriate.

1. A dilatable balloon implant, characterized in that it is designed to provide limited liquid permeability.

2. The implant according to claim 1, characterized in that a balloon of the implant is made of a material that provides limited liquid permeability.

3. The implant according to claim 2, characterized in that the material is a textile tissue or fabric.

4. The implant according to claim 3, characterized in that the material is elastic.

5. The implant according to claim 4, characterized in that the material is a nylon tissue.

6. The implant according to claim 5, characterized in that the textile material conforms to DIN Standard 53861-3.

7. The implant according to claim 2, characterized in that the material is inherently liquid impermeable and provided with pores.

8. The implant according to claim 7, characterized in that the pores account for up to 5% of the balloon surface.

9. The implant according to claim 7, characterized in that the material is stippled latex.

10. The implant according to claim 2, characterized in that the material used for the balloon has a permeability level to medical bone filler material of 0.05 to 6%.

11. The implant according to claim 1, characterized in that the balloon implant is preshaped in such a manner that it assumes a predetermined shape when dilated.

12. The implant according to claim 1, characterized in that the implant is provided with a means for joining, in a separable and conductive manner, with an introduction sleeve (2).

13. The implant according to claim 1, characterized in that it is provided, at its proximal open end, with a thinner wall area serving as a predetermined breaking point.

14. The implant according to claim 1, characterized in that it is provided, at its proximal open end, with a means for closing a balloon lumen of the implant.

15. A vertobroplasty device equipped with a balloon having implant having limited liquid permeability, which is connected to the distal end of an introduction sleeve in such a manner that the interior of the implant communicates with the introduction sleeve lumen.

16. A device according to claim 15, characterized in that the implant is joined to the introduction sleeve by means of a form-fit joining technique, such as gluing, clamping, expanding or bolting.

17. A device according to claim 15, characterized in that the connection between the introduction sleeve and the balloon implant is separable.

18. A device according to claim 15, characterized in that the connection between the introduction sleeve and the balloon implant is inseparable and that the balloon implant is provided with a predetermined breaking point.

19. A device according to claim 15, characterized in that the open end of the balloon implant is attached to the inner circumference of the introduction sleeve.

20. A device according to claim 15, characterized in that the open end of the balloon implant is pulled over the distal end of the introduction sleeve and attached to the outer circumference of the introduction sleeve.

21. A device according to claim 15, characterized in that the introduction sleeve is provided, at its distal end, with an additional means for securing the balloon implant in place.

22. A device according to claim 15, characterized in that the introduction sleeve is made of medical grade steel.

23. The implant according to claim 1, characterized in that the material used for the balloon has a permeability level to medical bone filler material of 1 to 4%.

24. The implant according to claim 1, characterized in that the material used for the balloon has a permeability level to medical bone filler material of 2 to 3%.
